(19) United States
(12) Patent Application Publication
(10) Pub. No.: US 2018/0361000 A1
(43) Pub. Date: Dec. 20, 2018

(54) DEXTRAN NANOPARTICLES FOR MACROPHAGE SPECIFIC IMAGING AND THERAPY

(71) Applicant: The General Hospital Corporation, Boston, MA (US)

(72) Inventors: Ralph Weissleder, Peabody, MA (US); Edmund J. Keliher, Topsfield, MA (US); Matthias Nahrendorf, Boston, MA (US)

(73) Assignee: The General Hospital Corporation, Boston, MA (US)

(21) Appl. No.: 16/060,812

(22) PCT Filed: Dec. 9, 2016
(86) PCT No.: PCT/US16/66003

§ 371 Date: Jun. 8, 2018

Related U.S. Application Data
(60) Provisional application No. 62/266,480, filed on Dec. 11, 2015.

(51) Int. Cl. A61K 51/12; A61K 51/06; A61B 6/03; A61B 5/055

(52) U.S. Cl. CPC A61K 51/1244; A61K 51/065; C08B 37/0021; A61B 5/055; A61B 6/037

(57) ABSTRACT

This disclosure relates to specific nanometer-sized nanoparticles made from unmodified dextran (DNPs), DNP conjugates, and related compositions and methods of use.

### CLAIM OF PRIORITY

This application claims the benefit of U.S. Provisional Patent Application Ser. No. 62/266,480, filed on Dec. 11, 2015. The entire contents of the foregoing are hereby incorporated by reference in their entireties.

### FIELD

This disclosure relates to dextran nanoparticles, dextran nanoparticle conjugates, and related compositions and methods of use.

### BACKGROUND

Macrophages are white blood cells that are produced by the differentiation of monocytes after they enter into tissues. The primary role of macrophages is to phagocytose pathogens and cellular debris. Given this role, macrophages are recruited to areas of tissue injury, and can further act to stimulate the recruitment of lymphocytes and other immune cells to these areas. Due to macrophages' widespread distribution throughout the body and their involvement in many different diseases, information regarding their total mass, relative numbers at different sites, as well as their mobilization and flux rates in different tissues, can be useful for a variety of purposes.

### SUMMARY

This disclosure relates to specific nanometer-sized nanoparticles made from unmodified dextran (DNPs), DNP conjugates, and related compositions and methods of use. These new DNPs are prepared from non-toxic materials and are suited for in vitro and in vivo uses, including for diagnostic and therapeutic uses in human and animal subjects. The new nanometer sized DNPs provide uniquely rapid pharmacokinetics and renal clearance, as opposed to the reticuloendothelial system (RES) clearance by mononuclear phagocytes of the liver (e.g., Kupffer cells) and yet are taken up selectively by macrophages compared to other immune cells. These characteristics make the new DNPs ideal for use in methods for ultra-fast (i.e., in less than one hour) targeting of macrophages with reporter groups, e.g., nuclear or fluorescent reporter groups, for imaging and diagnosis, and for delivery of active agents, e.g., drugs, small molecules, oligonucleotides, or proteins, to macrophages, e.g., macrophages resident in healthy tissue as well as macrophages recruited from the bloodstream (as monocytes) to injured or diseased tissue (such as in the heart after a heart attack or other organs after an ischemic incident, or in tumors or infected tissue), as well as for delivery of active agents to the kidneys for treatment of renal diseases.

In one aspect, the disclosure relates to nanometer-sized dextran nanoparticles (DNPs) including a plurality of carboxymethyl dextran polymer chains that are cross-linked by lysine. Embodiments can include one or more of the following features.

The DNPs can have an average particle size of between about 3 nm and about 15 nm, e.g., between about 3 nm and about 10 nm, about 3 nm and about 7 nm, and about 4 and about 6 nm. The DNPs further can include one or more different functional groups that link the DNPs to one or more different types of active agents. The functional groups can be, for example, an azide functional group, a sulfonate functional group, an amino, —NHC(O)(CH₂)ₙC(O)—, a carboxy group, or a sulfhydryl group. The active agents can be, for example, a radiolabel (radioactive isoptope) such as ¹⁸F, e.g., in the form of —(CO)ₙ—¹⁸F, wherein “n” can be any number between 0 and 10, e.g., 1, 2, 3, 4, 5, 6, 7, 8, 9, or 10. The active agent can also be other radioactive isotopes, fluorophores, such as VivoTag-S® 680 (VT680, Perkin Elmer, Waltham, Mass.), VivoTag-S® 750 (VT750, Perkin Elmer, Waltham, Mass.), BODIPY® FL (GE Life Technologies, Pittsburgh, Pa.), and BODIPY® 630 (GE Life Technologies, Pittsburgh, Pa.), or drugs such as doxorubicin or a large variety of anti-tumor, anti-inflammatory, or macrophage reprogramming drugs, e.g., in the form of small molecules, nucleic acids (such as RNAi or antisense constructs), peptides, proteins, or other biological macromolecules.

In another aspect, the disclosure relates to DNP compositions having a plurality of the DNPs described herein. In general, the average largest diameter of the DNPs is between about 3 nm and about 15 nm. In some embodiments, more than 95% of the DNPs in the composition have a diameter between about 3 nm and about 15 nm, e.g., between about 4 nm and about 7 nm. The diameter can be the largest diameter or the average diameter of each DNP.

In another aspect, the disclosure relates to in vivo methods of imaging macrophages in a subject. These methods include administering to the subject an effective amount of the DNPs described herein. An effective amount is a number of DNPs required to achieve a visible image and depends on the imaging modality used as well as the size and weight of the subject. The DNPs used in these methods include one or more imaging agents linked to the nanoparticles. After a suitable waiting period, e.g., 1 to 4 hours, the imaging agent can be imaged, e.g., viewed and/or recorded and/or analyzed, using an imaging technique, in a region of the subject in which macrophages have accumulated. The imaging technique can be, for example, positron emission tomography (PET), PET-computed tomography (PET/CT), PET-magnetic resonance imaging (PET/MRI), or fluorescence molecular tomography-CT (FMT-CT), as described herein.

In another aspect, the disclosure relates to methods of delivering one or more active agents, such as therapeutic agents, to a target site in a subject. In these methods, an effective amount of the DNPs described herein is administered to the subject. The DNPs include a therapeutic agent, e.g. doxorubicin or any of a wide variety of drugs, which is linked to the DNPs via a functional group or via a linker that serves to bind the therapeutic agent to a functional group on the DNPs. An effective amount is a number of DNPs including the active agents that when accumulated in a target region of the subject provide a desired effect, e.g., a therapeutic effect.

The new DNPs and the methods of use described herein provides several benefits and advantages. First, the DNPs with the appropriate reporter groups provide a new positron emission tomography (PET) imaging agent with sufficient specificity for tissue resident macrophages. Second, the nanometer-sized macrophage-targeted DNPs with narrow size distributions as described herein exhibit rapid pharmacokinetics and renal clearance, making them useful as imaging agents that are well suited for a fast and safe diagnostic use. Third, the ultra-fast pharmacokinetics enable imaging faster imaging than other imaging agents previously developed, making the new compositions and methods safer for patients. Fourth, PET imaging for tissue resident macrophages has important applications for imaging in oncology and cardiovascular diseases (i.e. atherosclerosis, transplants, myocardial infarction, and chronic heart failure) and drug testing. Fifth, the nanometer-sized macrophage-targeted DNPs have a short half-life (about 30 minutes in human subjects) and thus enable imaging with radioactive labels with only minimal exposure of the subject to the radioactive material.

As used herein, the term “nanometer-sized” when used to describe the DNPs means within a size range of about 3 nm to 15 nm.

As used herein, by “linked” is meant covalently or non-covalently associated. By “covalently” linked to a nanoparticle is meant that an agent is joined to the nanoparticle either directly through a covalent bond or indirectly through another covalently bonded agent. By “non-covalently bonded” is meant joined together by means other than a covalent bond (for example, by hydrophobic interaction, Van der Waals interaction, and/or electrostatic interaction).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of one example of a synthetic pathway to prepare the new DNPs from carboxymethyl (CM) dextran using lysine as a cross-linking agent.

FIG. 2 is a schematic diagram of one example of a transverse flow filtration system for refining the size of DNP.

FIG. 3 is a schematic diagram of one example of a synthetic pathway to prepare ¹⁸F-DNP from carboxymethyl (CM) dextran, lysine, azides, and 3-(2-(2-(2-[18F]-fluoroethoxy)ethoxy)ethoxy)-prop-1-yne (18F-P3C#C).

FIGS. 4A to 4C are a series of schematic diagrams of synthetic pathways to incorporate various functional groups onto and into the new DNPs.

FIG. 5 is a schematic diagram of one example of a synthetic pathway to prepare CMDex-LY and DNP-LY from carboxymethyl (CM) dextran and lucifer yellow carbohydrazine (LYCH).

FIG. 6 is a schematic diagram of one example of a synthetic pathway to prepare DNP-doxorubicin from carboxymethyl (CM) dextran, lysine, Boc-hydrazine, and doxorubicin.

FIG. 7A is a graph that shows the result of dynamic light scattering for the DNP prepared based on the method described in Example 1. FIG. 7B is a graph that shows the result of size-exclusion chromatography for the DNP prepared based on the method described in Example 1.

FIG. 8A is a series of graphs showing the distribution of fluorescently labeled DNP-VT680 in various leukocytes in mice, including macrophages, measured by flow cytometry.

FIG. 8B is a graph that shows the blood half-life of DNP in mice.

FIG. 8C is a graph that shows the biodistribution of ¹⁸F-DNP in mice.

FIG. 8D is a graph of autoradiography of an infarct area.

FIG. 8E is a graph that shows pale infarct area with 2,3,5-Triphenyl-2H-tetrazolium chloride (TTC) staining (viable heart muscle stains deep red with TTC while infarctions stain a pale red or pink).

FIG. 8F is a graph that shows the result of scintillation counting of control versus infarcted hearts.

FIG. 8G is a series of in vivo PET/MRI graphs showing higher ¹⁸F-DNP uptake in the infarct on day 6 than day 2, reflecting increasing infarct macrophage numbers.

FIG. 9A is a series of PET graphs of a baboon (Papio anubis) showing rapid renal clearance of ¹⁸F-DNP from the blood pool.

FIG. 9B is a graph that shows blood counts of ¹⁸F-DNP at different time points.

FIG. 9C is a PET graph that shows the distribution of ¹⁸F-DNP 90 minutes after the injection.

FIG. 9D is a combined PET and MRI image of the same baboon as in FIG. 8C.

FIG. 10 is a combined PET-CT image of a mouse with bilateral flank tumors.

FIG. 11 is a graph showing the hydrolysis of DNP-LY under different pH conditions.

FIG. 12A is a schematic diagram of one example of a synthetic scheme of labeling DNP with ⁶⁸Ga.

FIG. 12B is a graph showing radiochemical purity of ⁶⁸Ga labeled DNP.

FIG. 12C is a graph showing bio-distribution of ⁶⁸Ga labeled DNP in wild type mice.

FIG. 12D is an autoradiography image of aorta harvested from ApoE−/− mice with atherosclerosis.

FIG. 12E is a graph showing in vivo PET imaging with strong PET signal in kidneys.

FIG. 12F is an axial view of PET imaging showing increased PET signal in the aortic root of an ApoE−/− mouse with atherosclerosis.

### DETAILED DESCRIPTION

This disclosure relates to new nanometer-sized dextran containing nanoparticles, dextran nanoparticle conjugates, and related compositions and methods of use.

The specific nanometer-sized dextran nanoparticles described herein (DNPs), upon systemic administration, are readily engulfed by mononuclear phagocytic cells while the remainder are rapidly excreted. Due to macrophages' involvement in many different diseases, the DNPs can thus be used as imaging probes for examining diseased tissue in humans as well as targeted delivery vehicles to direct active agents to macrophages in diseased or injured tissues throughout the body. In addition, the rapid renal clearance of the new DNPs enables them to be used as delivery vehicles to direct active agents such as drugs and other therapeutic agents to the kidneys.

While some nanoparticles have been developed for magnetic resonance imaging (MRI), these particles are typically much larger to carry a magnetic payload. For instance, Feramoxytol (Feraheme) has a mean hydrodynamic diameter of 30 nm (Simon, G H et al. Invest. Radiol. 2006, 41, 45-51.). Other therapeutic nanoencapsulating materials, such as the polylactic-co-glycolic acid-polyethylene glycol (PLGA-PEG), can form particles with mean diameter sizes of about 150 nm (Farokhzad et al., Proc. Natl. Acad. Sci. USA., 103, 6315-6320, 2006). These large-sized particles have long blood half-lives in humans, 12-24 hours, and eliminated by hepatobiliary clearance from the blood. Therefore, patients are exposed to these materials for extended periods of time. In addition, it is often complicated to quantify exact nanoparticle concentrations by MRI in vivo in different organs, particularly in bone marrow and lung tissue.

In contrast, the present disclosure describes novel labeled, e.g., radiolabeled, DNPs that have interesting clinical applications. In general, it is possible to administer the new radiolabeled DNPs at much lower concentrations than their magnetic counterparts, making them much safer for human use. The present disclosure also describes a new ¹⁸F-labeled DNP imaging agent for PET imaging with good specificity for tissue-resident macrophages. This is not intuitive since ¹⁸F decays with a radioactive half-life of 109 minutes and macrophage accumulation usually occurs beyond this time frame. The nanometer-sized macrophage-targeted DNPs also have a narrow size distribution of 3 nm to 15 nm, e.g., 3 nm to 10 nm, 3 nm to 7 nm, or 4 nm to 6 nm, and thus exhibit rapid pharmacokinetics and renal clearance, making the new DNPs well suited for a fast and safe clinical use.

### Methods of Making Carboxymethyl Dextran Nanoparticles

DNP Synthesis

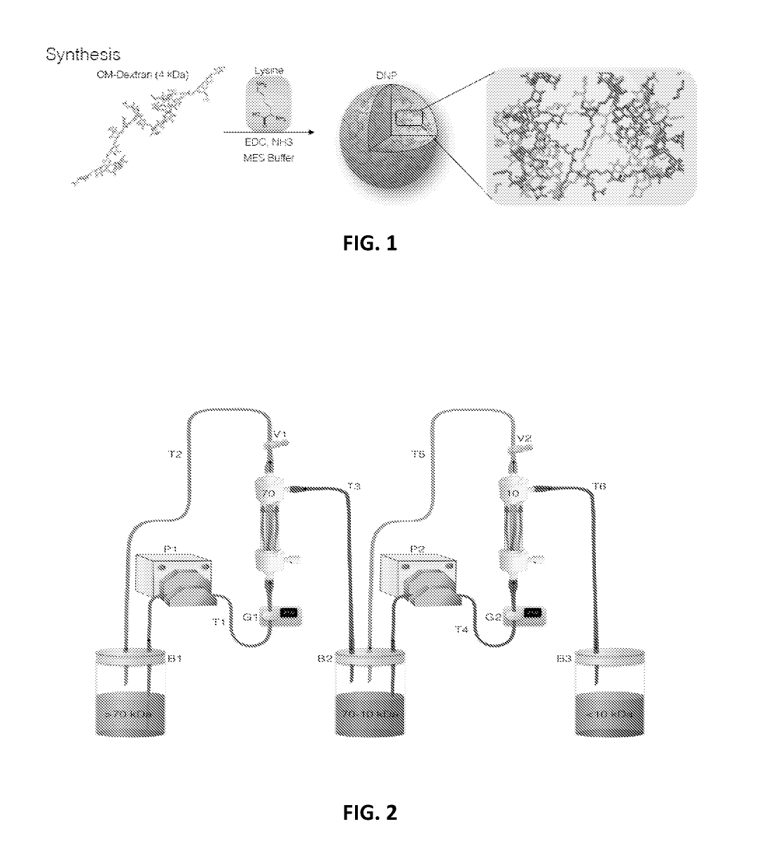

The new DNPs are ideally synthesized from carboxymethyl-dextran (CM-dextran) using a physiologically acceptable cross-linking agent such as lysine (FIG. 1). An appropriate amount of a crosslinking agent such as lysine is mixed with CM-dextran, and one or more carboxyl activating agents, e.g., N-(3 dimethylaminopropyl)-N′-ethylcarbodiimide (EDC) hydrochloride, and N-hydroxysuccinimide (NHS), dissolved in a buffer, such as 2-(N-morpholino) ethanesulfonic acid (MES). After stirring for a sufficient time, the mixture is diluted with ethanol. The resulting suspension is vortexed and centrifuged to form a pellet that contains the desired CM-dextran nanoparticles. The ethanol solution is decanted off, and each pellet is dissolved in H₂O and passed through a filter, e.g., a 0.22 μm filter, to obtain a sterile crude product. The combined crude filtrate is subjected to size-exclusion chromatography or flow filtration as described below to refine the particle size distribution to about 4 to 6 nm.
Size Refinement

The particle size of the crude product can be refined, for example, by size-exclusion chromatography, transverse flow filtration, diafiltration, or ultrafiltration.

The particle size of the crude product can be refined by size-exclusion chromatography (SEC) as follows. In a typical SEC method, the crude product is loaded onto a column, such as a PD-10 column (GE Life Sciences) or Superdex® 200 column (GE Life Sciences), the eluent is then collected in constant volumes, known as fractions. The relevant fractions containing the nanoparticles with desired size are collected.

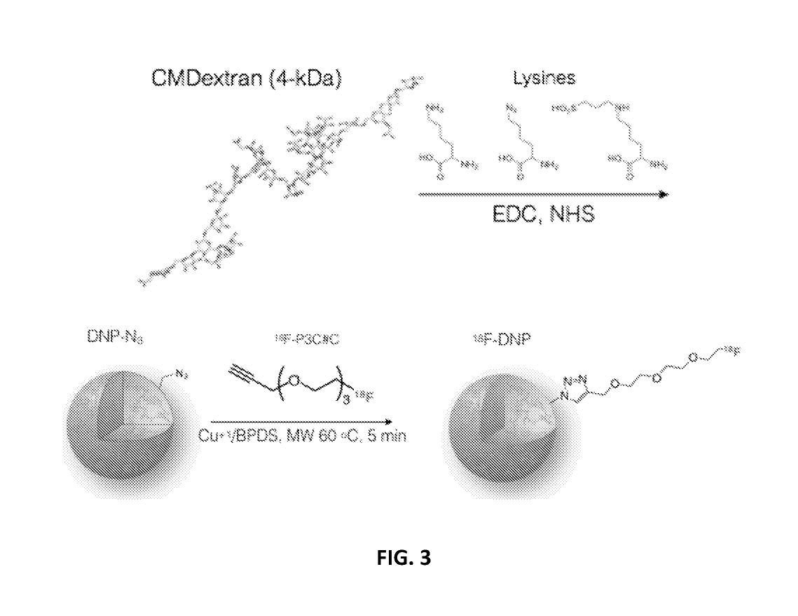

The crude product can also be refined by transverse flow filtration using a system such as the one shown in FIG. 2. In this system, the crude product is forced through a 70-kDA tangential flow filtration (TFF) filter and a 10-kDA TFF filter under pressure. The final contents are then passed through centrifuge filters and concentrated by centrifugation.

In the particular embodiment shown in FIG. 2, the crude product can be added to bottle B1, and diluted, e.g., to a volume of 1 L, using a diluent such as H₂O (e.g., MilliQ H₂O filtered through 0.22 μm filter). The bottle B1 is then connected to the system, and a peristaltic pump P1 forces the crude product through a 70-kDA tangential flow filtration filter. A back-pressure valve V1 is adjusted to an appropriate pressure, and the pressure gage G1 is monitored such that it does not exceed the set pressure, e.g., 25 psig. Once the volume of bottle B2 reaches the bottom of a feed tubing for the 10-kDA TFF filter, a second pump P2 is turned on, forcing the filtrate through a 10-kDA TFF filter into collection bottle B3.

A second back-pressure valve V2 is similarly adjusted such that an appropriate pressure is observed at the pressure gage G1. The pressure is monitored such that it does not exceed a desired pressure, e.g., 25 psig. When the volume remaining in bottle B1 is less than a certain level, pump P1 is turned off. Collection bottle B3 is removed, and replaced with another collection bottle B3. The crude product in bottle B1 is then diluted to an appropriate volume, and the pumps P1 and P2 are turned on. The pressure at gauges G1 and G2 are monitored such that it does not exceed a desired pressure, e.g. 6.5 psig. The size-refinement steps are repeated for a few times. The contents in these collection bottles B3 are then passed through 10-kDa molecular weight cut-off (MWCO) 50 ml centrifuge filters and concentrated by centrifugation at approximately 2500 g. The final product contains the CM-dextran nanoparticles in a size range of about 4 to 6 nanometers.
Surface Modification

The surface of DNP can be further modified.

To lower the surface charge (zeta-potential), all amines on DNP can be capped with succinic anhydride. A lower surface charge will aid in renal clearance.

To form aldehydes in/on the particles to allow conjugation of active agents, such as drugs, fluorophores, or other imaging agents for release through a pH dependent hydrolysable condensation reaction, oxidizing agents, such as sodium periodate (NaIO₄), can be used to react with dextran to form aldehydes. Escalated amounts of oxidation are used to show that different amounts of drug can be conjugated to DNP. In one particular embodiment, the fluorophore is Lucifer Yellow (LY).

To form a non-reversible conjugation, DNP with azide can be reacted, for example, with Bicyclononyne-fluorophore (BCN-VT680XL).
DNP Labeling

The DNPs can be labeled with a variety of reporter groups, such as fluorescent or nuclear reporter groups.

To make the particle fluorescent, so that the particle can be used for fluorescence microscopy (in vitro screens or in vivo imaging), immuno-fluorescent histology, a percentage of amines on DNP is reacted with a fluorochrome. Following attachment of fluorochrome, the remaining amines were capped with succinic anhydride. If desired other modification step could be done before the succinic anhydride capping of remaining amines. For example, one useful fluorescent reporter group is the fluorophore VivoTag® 680 (VT680, Perkin Elmer, Waltham, Mass.) to form DNP-VT680. Some other examples of fluorescent reporter groups include VivoTag® 750 (VT750, Perkin Elmer, Waltham, Mass.), BodipyFL® (GE Life Technologies, Pittsburgh, Pa.), and Bodipy®630 (GE Life Technologies, Pittsburgh, Pa.).

In a typical fluorophore conjugation reaction, DNP-amine is diluted with 2-(N-morpholino) ethanesulfonic acid (MES) buffer (pH 6) and then treated with triethylamine (Et₃N) and VT680-NHS (dissolved in dimethylformamide (DMF)). The mixture is then shaken for sufficient time at room temperature. The reaction mixture is then loaded onto a size-exclusion chromatography (e.g., PD-10 cartridge) and eluted with MilliQ water. Appropriate fractions are combined and concentrated using 10-kDa MWCO filters.

To incorporate azides for labeling via rapid click chemistry, this solution is diluted with MES buffer and treated with Et3N and azidoacetic acid NHS ester (in dimethyl sulfoxide (DMSO)) and then shaken at for sufficient time at room temperature. This reaction mixture is loaded onto a size-exclusion chromatography (e.g., PD-10 cartridge) and eluted as described above. To end-cap remaining amines, the solution is diluted with MES buffer and treated with Et₃N and succinic anhydride.

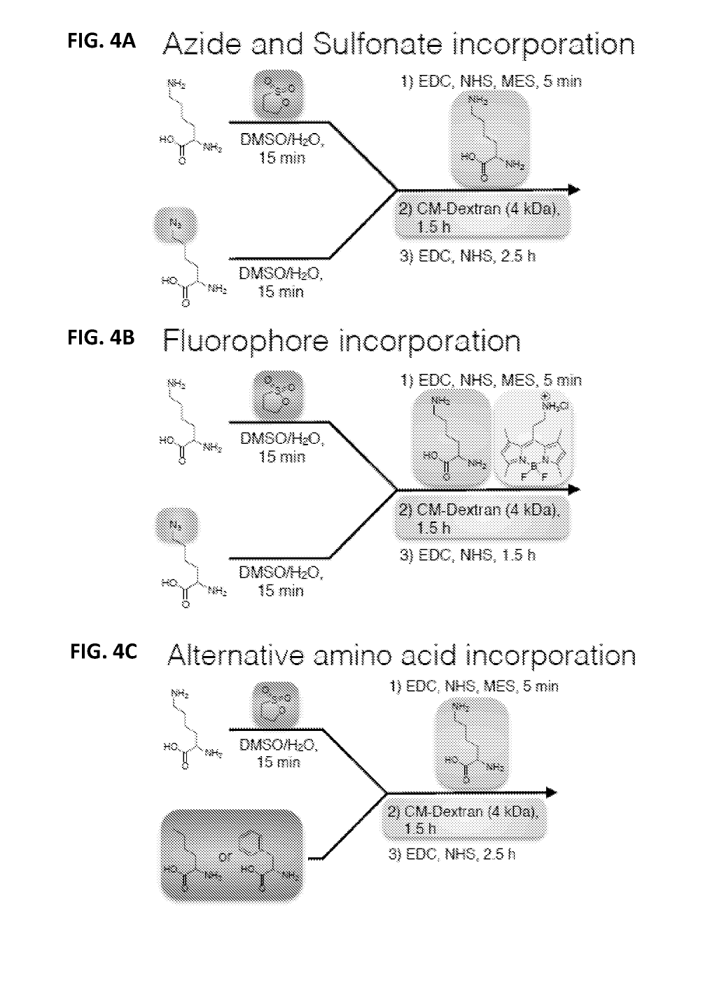

In some embodiments, ¹⁸F labeling of DNP is achieved by copper catalyzed azide/alkyne click chemistry for bioconjugation (FIG. 3). First, an ¹⁸F-prosthetic group 3-(2-(2-(2-[¹⁸F]-fluoroethoxy)ethoxy)ethoxy)-prop-1-yne (¹⁸F-P3C#C) is synthesized. This is combined with the azido-DNP in the presence of copper catalyst and heated to 60° C. for 5 minutes. After heating, the mixture is subjected to size-exclusion chromatography (SEC) for purification. This reaction is described in detail in Devaraj et al., Bioconjug Chem, 20, 397-401 (2009), which is incorporated herein by reference in its entirety. Analysis by radio thin-layer chromatography (TLC), and analytical radio-SEC can be used for quality control.

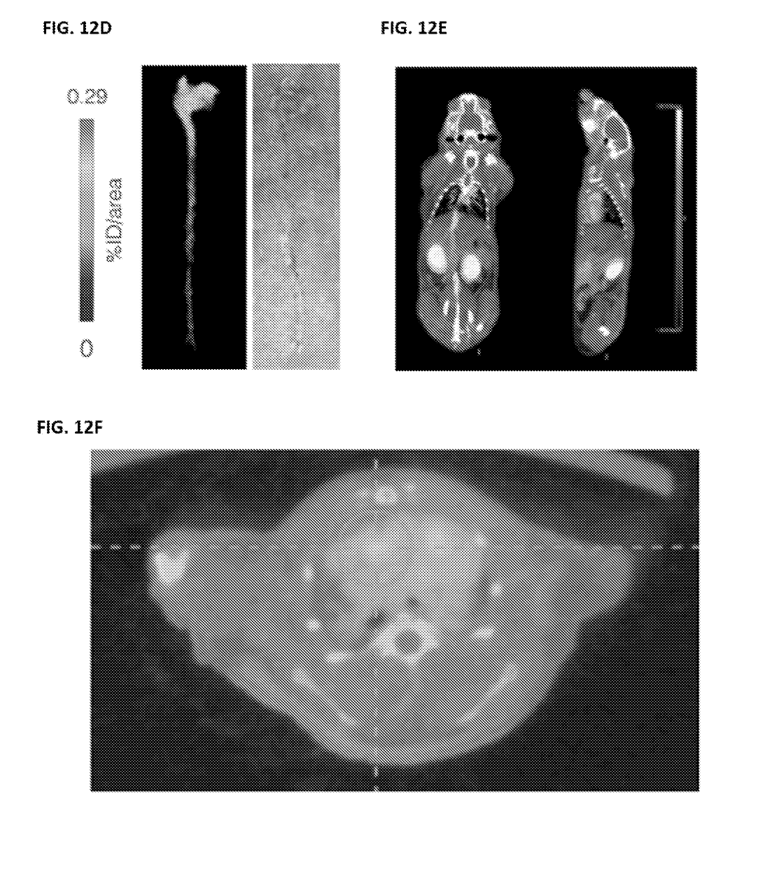

In some embodiments, ⁶⁸Ga labeling can be achieved by Cu-free strain-promoted alkyne-azide cyclization (SPAAC) (FIG. 12A). In some embodiments, DNP-azide is dissolved in deionized water. NODA-GA is linked to DNP-azide via Cu-free strain-promoted alkyne-azide cyclization (SPAAC). Then, BCN-NODA-GA (CheMatech, Dijon France, Cat. # C131, bicyclononyne-1,4,7-triazacyclononane, 1-glutaric acid-4,7-acetic acid) is added to the DNP aqueous solution followed by agitation at room temperature overnight. Unreacted BCN-NODA-GA can be removed by PD-10 column purification. Dextran-positive fractions are combined and centrifuged (e.g., with 10 kDa MWCO). ⁶⁸Ga is added to NODA-DNA at an optimal condition (e.g., pH 6, 80° C., 10 min). The NODA-DNP is then labeled with ⁶⁸Ga. In some embodiments, the NODA-DNP solution can be lyophilized, and stored for future use.
DNP Conjugates and Other Modifications

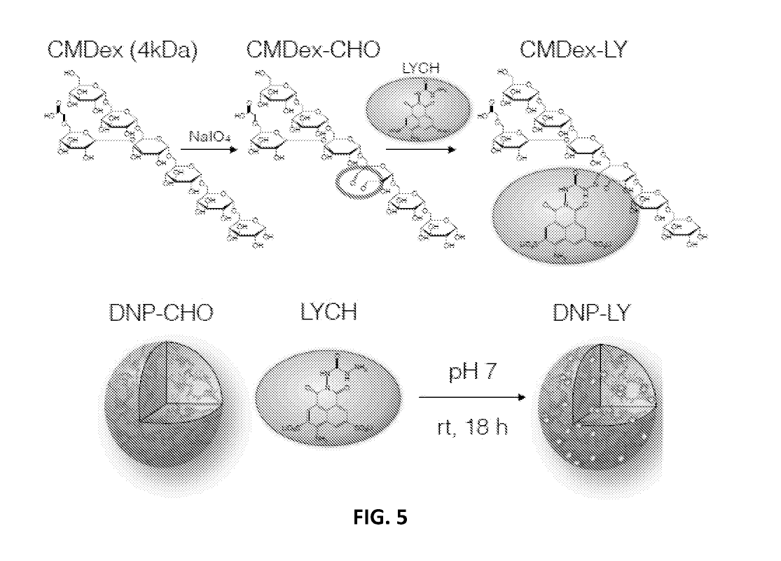

Any amine or carboxylic acid active molecules can be incorporated into the base DNP during the synthesis. In some embodiments, azides, sulfonates, fluorophores and amino acids or amino acid derivatives can be incorporated into the base DNP. A synthetic scheme for azide or sulfonate incorporation is shown in FIG. 4A, while FIG. 4B shows a synthetic scheme for fluorophore incorporation. An alternative amino acid incorporation scheme is shown in FIG. 4C. The amino acids and amino acid derivatives include, but are not limited to, azido-lysine, phenylalanine, leucine, histidine, arginine, aspartic acid, tyrosine, and tryptophan.

Further, by increasing the amount of azides in the synthesis reaction, the amount of reactive azides in the particle can be increased without affecting the key characteristics of the particle. Any changes to particle size or chemistry may affect the way the particle interacts in vivo, so analyzing the particle after each change is necessary. The FDA measures dosage by milligram of particle, so increasing this ratio may be important for clinical use. In the clinical setting, significantly higher amounts of radioactivity can be used.

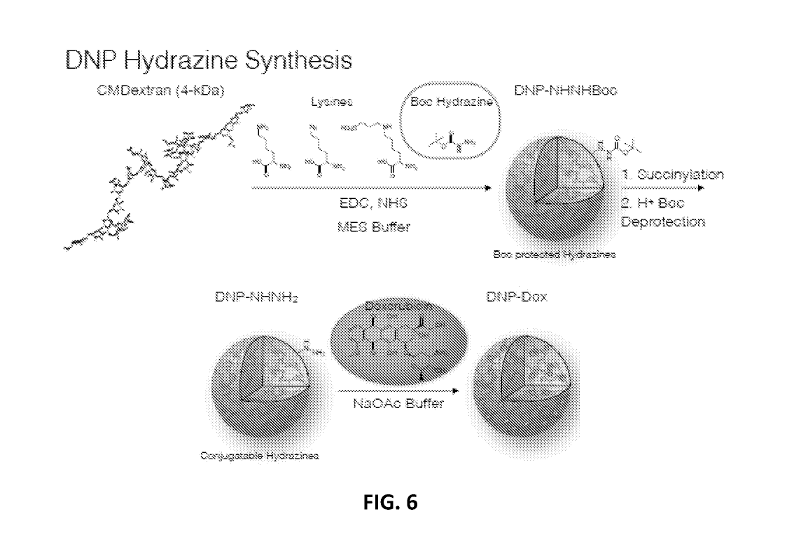

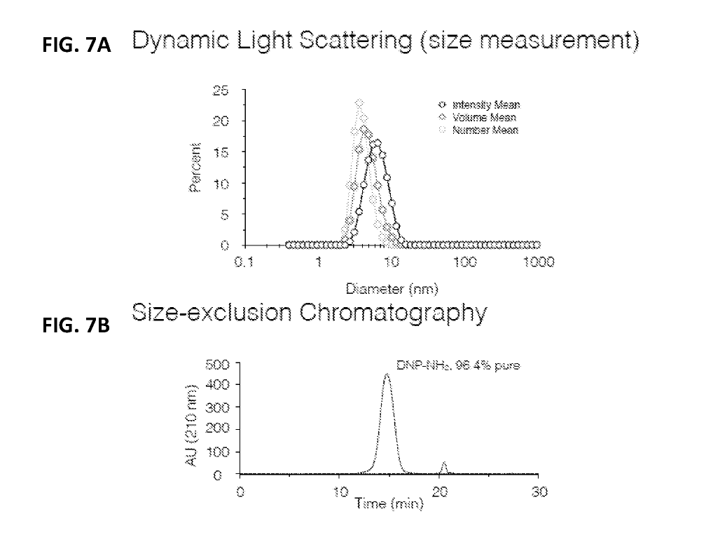

Experiments are also performed to determine whether the DNP particle can deliver drug to a target site. For testing, lucifer yellow carbohydrazine (LYCH) and the chemotherapeutic doxorubicin are used. DNP is conjugated with Lucifer Yellow through Lucifer Yellow carbohydrazine. DNP is first treated with NaIO₄ and then react with Lucifer Yellow carbohydrazine (FIG. 5). Regarding DNP-doxorubicin, DNP is conjugated with doxorubicin based on the method as shown in FIG. 6.
Characterization and Quality Control

Experiments can be performed to determine the characteristics of DNP.

For example, the particles can be characterized by dynamic light scattering (DLS) and zeta potential to determine size and surface charge. DLS and size-exclusion chromatography serve as quality control and ensure nanoparticle integrity and size uniformity.

The amount of reporter group, such as VT680, conjugated to each nanoparticle can also be analyzed, e.g., by use of the NanoDrop® system (Thermo Scientific micro-volume UV-Vis spectrophotometers and fluorospectrometers) to quantify total moles of the reporter group conjugated to the nanoparticles.

For mass quantification, a nanoparticle sample can be frozen in dry ice, then lyophilized. The weight of lyophilized sample can then be measured.

For determining the weight contribution of carboxymethyl dextran to the nanoparticles, a nanoparticle sample with different concentrations can be mixed with phenol and concentrated H₂SO₄ for a sufficient time at room temperature. The absorbance at 490 nm of each mixture was determined using a NanoDrop system. Linear regression of series data is performed, and the results can be used as a standard to estimate the carboxymethyl dextran content of the nanoparticle samples.

For quantifying the amine content of the nanoparticles, a dilution series of glycine stock solution and a dilution series of aminodextran stock solution are prepared and mixed. Bicarbonate and 2,4,6-Trinitrobenzenesulfonic acid solution (TNBS) solution are further added and mixed. The absorbance at 420 nm of each mixture was determined using UV spectrophotometer system (e.g., NanoDrop). Linear regression of dilutions series data is performed, and the results are used as a standard to estimate the amine content of the nanoparticle samples.

For quantifying the azide content of the nanoparticles, an aliquot of a nanoparticle sample can be mixed with fluorescein-5-alkyne (FAM-5C#C, Lumiprobe, Hallandale Beach, Fla.) solution in DMF, 4,7-diphenyl-1,10-phenanthrolinedisulfonic acid (BPDS) solution, and Cu+1 solution in MeCN. The mixture is flushed with argon for a short period of time. The sample is then microwave irradiated (60° C., 30 W for 5 minutes). The reaction mixture is loaded onto a PD-10 column and eluted. Fractions are collected. The fractions that are yellow in color are combined and concentrated using 10 kDa MWCO filters. The material collected from the 10 kDa filters is recovered and the final volume recorded. The absorbance of FAM-5C#C conjugated to the DNP at 485 nm are measured using the NanoDrop. The concentration of FAM-5C#C from the absorbance is calculated using the equation C (aliquot)=A/eb (where e=extinction coefficient, in this case 80000).

### Methods of Use

The new DNPs can be used in a variety of targeted imaging (diagnostic) and targeted delivery (therapeutic) methods. In each case, the targeted region in a subject, e.g., a human or animal subject, is either an area of macrophage accumulation or the kidneys.
Positron Emission Tomography-Computed Tomography (PET/CT)

Positron emission tomography-computed tomography (PET/CT) is a medical imaging technique using a device that combines in a single gantry system both a positron emission tomography (PET) scanner and an x-ray computed tomography (CT) scanner. The new DNPs appropriately labeled can be used to generate PET images of accumulations of the DNPs, such as in macrophages that have accumulated in a diseased or injured tissue, or in the kidneys, even without macrophage uptake. Useful reporter groups include radioactive isotopes, such as ¹¹C, ₁₃N, ¹⁵O, ¹⁸F, ⁶⁴Cu, ⁶⁸Ga, ⁸¹mKr, ⁸²Rb, ⁸⁶Y, ⁸⁹Zr, ¹¹¹In, ¹²³I, ¹²⁴I, ¹³³Xe, ²⁰¹Tl, ¹²⁵I, ³⁵S ¹⁴C, ³H.

Images acquired from both devices can be taken sequentially, in the same session, and combined into a single superposed (co-registered) image. Thus, functional imaging obtained by PET, which depicts the spatial distribution of metabolic or biochemical activity in the body can be more precisely aligned or correlated with anatomic imaging obtained by CT scanning Two- and three-dimensional image reconstruction may be rendered as a function of a common software and control system.

PET/CT scans can be used to diagnose a health condition in human and animal subjects. In a typical setting of performing PET/CT scans on for research animals such as mice, rats, and even larger animals, the animals are anesthetized, e.g., by isoflurane, prior to imaging, and anesthesia is maintained during the process. CT acquisition precedes PET and lasts approximately 4 minutes, acquiring 360 cone beam projections with a source power and current of 80 keV and 500 μA, respectively. Projections are reconstructed into three-dimensional volumes. The imaging bed then moves into the PET gantry. In one embodiment, the radioactively labeled DNPs are injected at the beginning of PET acquisition via tail vein catheter, which is set up prior to imaging. In some other embodiments, labeled DNP are similarly injected prior to PET-CT/PET-MR/FMT-CT, and imaging starts 1-4 hours post injection. A high-resolution Fourier re-binning algorithm is used to re-bin sinograms, followed by a filtered back-projection algorithm for reconstruction. The reconstructed PET image, through dynamic framing of the sinograms, is composed of a series of 1, 3, and 5 minute frames. PET and CT reconstructed images are then fused using Inveon Research Workplace (IRW) software (Siemens). The described method is useful for diagnosing many diseases, such as cancers, (e.g., lung, brain, pancreatic, melanoma, prostate, colon cancers), cardiovascular disease (e.g., myocardial infarction, atherosclerosis), autoimmune diseases (e.g., multiple sclerosis, diabetes, irritable bowel syndrome, Celiac disease, Crohn's disease), and pelvic inflammatory disease.
Positron Emission Tomography-Magnetic Resonance Imaging (PET-MRI)

Positron emission tomography-magnetic resonance imaging (PET-MRI) is a hybrid imaging technology that incorporates magnetic resonance imaging (MRI) soft tissue morphological imaging and positron PET functional imaging. PET/MRI scans can be used to diagnose a health condition in humans and animals, e.g., for research and agricultural purposes. The new DNPs when appropriately labelled can be used in PET/MRI.

In a typical setting of performing PET/MRI scan on mice, PET/MRI registration and fusion are facilitated by a custom-made mouse bed. The method is described in detail in Lee et al., J. Am. Coll. Cardiol., 59:153-63 (2012), which is incorporated herein by reference in its entirety. For imaging a particular organ, such as the heart, a fusion approach is implemented using external fiducial landmarks provided by a “vest” optimized for the particular organ, e.g., for cardiac imaging. The vest surrounds the subject's chest to create a frame that follows minor movements due to transfer between scanners or light anesthesia. The tubes are filled with 15% iodine in water, rendering them visible in MRI. Subject motion is minimized with an imaging bed that can be used in both imaging systems.

The described methods are useful for diagnosing many diseases, such as cancers, (e.g., lung, brain, pancreatic, melanoma, prostate, colon cancers), cardiovascular disease (e.g., myocardial infarction, atherosclerosis), autoimmune diseases (e.g., multiple sclerosis, diabetes, irritable bowel syndrome, Celiac disease, Crohn's disease), and pelvic inflammatory disease.
In Vivo Fluorescence-Molecular Tomography-Computed Tomography (FMT-CT)

FMT-CT imaging is performed at 680/700 nm excitation/emission wavelength in cohorts of mice at 2, 4, 8, 24, and 48 hours after injection of 2.5 nmol of respective fluorochrome using an FMT 2500 system (VisEn Medical, now Perkin-Elmer, Waltham, MA) with an isotropic resolution of 1 mm. Mice are anesthetized (Isoflurane 1.5%, O₂ 2L/min) during imaging with a gas delivery system integrated into the multimodal imaging cartridge that holds the mouse during FMT and CT imaging. This cartridge facilitates coregistration of FMT to CT data through fiducial landmarks on its frame. Total imaging time for FMT acquisition is typically 5 to 8 minutes. Data are postprocessed using a normalized Born forward equation to calculate three-dimensional fluorochrome concentration distribution. CT angiography immediately follows FMT to robustly identify the aortic root. The detailed method is described in Nahrendorf et al., Arterioscler Thromb Vasc Biol., 29:1444-1451 (2009), which is incorporated herein by reference in its entirety.
Monitoring Immuno Modulation

While macrophages play important roles in development, repair, regulation of homeostasis, and defense against infection, they can also turn against the host. For example, inflammatory macrophages likely promote disease in ischemic hearts. Organ ischemia triggers a controlled biphasic monocyte/macrophage response. An inflammatory “demolition” phase, during which inflammatory monocyte/macrophages remove dead cells and matrix, transitions towards a “reparative phase” on days 3/4 after ischemia. This transition is impaired by an overzealous supply of inflammatory monocyte-derived macrophages, leading to compromised resolution of infarct inflammation and heart failure. Research shows that the spleen as well as the bone marrow supply several hundred thousand monocytes each day after acute MI and that sympathetic nervous system activation is a key promotor of macrophage oversupply to cardiovascular organs. These concepts imply that macrophage monitoring during immuno-modulation is a valuable diagnostic tool.

In addition, as discussed in further detail below, promoting the transition from the inflammatory to the reparative monocyte/macrophage phase, e.g., “reprogramming” the macrophages, should reduce post-MI heart failure and improve long-term outcomes. Some strategies have been proposed to achieve this goal, either by blocking differentiation of monocytes into macrophages (silencing of M-CSF receptor, e.g., with macrophage-targeted in vivo RNAi) or by reducing the systemic supply of inflammatory monocytes (e.g., using a β3 adrenergic receptor blockade in the bone marrow).

Another prominent example of macrophage immune-modulation is tumor-associated macrophages (TAMs), which are commandeered by cancer cells to evade the host's defenses and promote tumor growth. TAMs are derived from circulating monocytes or resident tissue macrophages, which form the major leukocytic infiltrate found within the stroma of many tumor types. The function of TAMs is controversial. However, there is growing evidence for TAM's involvement in both pro-tumor (e.g., promotion of growth and metastasis through tumor angiogenesis) as well as anti-tumor (tumoricidal and tumorostatic) processes. The function appears to depend on the type of tumor with which they are associated. In some tumor types TAM infiltration level has been shown to be of significant prognostic value. TAMs have been linked to poor prognosis in breast cancer, ovarian cancer, types of glioma and lymphoma, but better prognosis in colon and stomach cancers. Therefore, macrophage monitoring using the new DNPs is a potential tool to evaluate the prognosis for a patient with tumors.
DNP Drug Delivery

The DNPs can be used to deliver an active agent such as a drug, a diagnostic agent, a therapeutic agent, an imaging agent, a small molecule, an oligonucleotide, a peptide, a protein, an antibody, or an antigen binding fragment to a target site. The goal is to prepare a DNP macrophage-specific active agent delivery platform, effectively turning the DNPs into a theranostic agent. These active agents can be either macrophage targeted or non-macrophage targeted, e.g., for use in cancer therapies, renal therapies, post-MI therapies, and to treat atherosclerotic plaque.

One useful method to incorporate active agents, such as drugs, into the DNPs to provide a sustained release upon injection, is the hydrazine-aldehyde conjugation method. In this method a hydrazone is formed to covalently bind the active agent to the DNPs. Once this conjugate is taken up by macrophages, the low pH in the endosome (pH of about 4-5) will result in hydrolysis of the hydrazone, releasing the bound active agent into the macrophage.

In certain embodiments, DNP-active agent conjugates are injected intravenously. The conjugates are distributed systemically and extravasate out of the blood vessels. Macrophages then take up the DNP-Active agent conjugates. The active agents, e.g., drugs, are released from the DNP inside the macrophages. For macrophage targeted therapies, the drug targets enzymes/ proteins inside/on the macrophage. For non-macrophage targeted therapies, the drug can migrate outside macrophages to neighboring cells.

In certain embodiments, DNP-active agent conjugates are injected intravenously or intraperitoneally. After administration, a period of time of at least about 1 hour, 1.5 hours, 2.0 hours, or 3.0 hours must pass before imaging is done to enable the macrophages to take up the DNPs.

In certain embodiments, macrophages can be extracted from blood samples and then mixed with the DNP-active agent conjugates and then re-administered to the patient.

In some embodiments, DNP-active agent conjugates can be used to treat cancers, such as breast cancer, ovarian cancer, glioma, lymphoma, colon cancer, and stomach cancer. The active agents for cancer treatment can be cytotoxic agents, cytostatic agents, growth inhibitors, CSF-1 inhibitors, etc.

In one embodiment, DNP-active agent conjugates are administered to a patient to treat glioblastoma multiforme (GBM). GBM is the most aggressive form of glioma. Patients respond minimally to currently used therapies, including surgery, radiation and chemotherapy. One challenge in treating GBM is substantial tumor-cell and genetic heterogeneity, leading to aberrant activation of multiple signaling pathways. Several approaches have been used to reprogram or ablate TAMs or to inhibit their tumor-promoting functions. One strategy is CSF-1R inhibition, which depletes macrophages and reduces tumor volume in several xenograft models. One potent CSF-1R inhibitor BLZ945 is described in Pyonteck S M, Akkari L, Schuhmacher et al., “CSF-1R inhibition alters macrophage polarization and blocks glioma progression,” Nature Medicine, 19(10):10.1038/nm.3337. doi:10.1038/nm.3337 (2013), which is incorporated herein by reference in its entirety.

Several other potential CSF-1R inhibitors to reprogram TAMs are described in WO 2012/151523 A1, entitled “CSF-IR Inhibitors for Treatment of Brain Tumors,” which is incorporated herein by reference in its entirety. These CSF-1R inhibitors can be linked to the DNPs described herein and be delivered to macrophages to treat various cancers include bone cancers and glioblastoma multiforme.

### EXAMPLES

The present disclosure further describes the following examples, which do not limit the scope of the invention described in the claims.

### Example 1

### Synthesis and Characterization of Nanometer Sized Carboxymethyl Dextran Nanoparticles

Experiments were performed to make and characterize nanometer sized carboxymethyl dextran nanoparticles. Experiments were also performed to label DNP with VT680 and with ¹⁸F.

### Methods

Synthesis of DNP Particles

Nanoparticles were synthesized from lysine and carboxymethyl-dextran (CM-dextran) according to the scheme in FIG. 1. The experiments were preformed based on the detailed procedure described below.

First, a crude product was synthesized by crosslinking dextran using carboxymethyl and lysine as crosslinkers.

320 mg of dry H-(L)-Lysine-OH, 320 mg of dry N-Hydroxsuccinymide, and 1 g of dry 1-Ethyl-3-(3-dimethylaminopropyl) carbodiimide were combined in a first 20 ml scintillation vial (labeled vial “A”).

550 mg of dry Carboxymethyl dextran (4-kDa), 65 mg of dry N-Hydroxsuccinymide, and 320 mg of dry 1-Ethyl-3-(3-dimethylaminopropyl) carbodiimide were combined in a second 20 ml scintillation vial (labeled vial “B”).

141 mg of H-(L)-Lysine(N3)-OH HCl, 640 μl of H₂O (MilliQ), and 5 μl of Triethylamine were combined in a 1.5 ml centrifuge tube (labeled tube “C”), and shaken for approximately 15 to 20 minutes.

32 mg of H-(L)-Lysine-OH, 120 μl of H₂O, 320 μl of DMSO, 5 μl of Et₃N, 320 μl of 1,3-propane sultone (500 mM in DMSO) were combined, in order, in a second 1.5 ml centrifuge tube (labeled tube “D”), and shaken for approximately 15 to 20 minutes.

5 ml of 50 mM 2-(N-morpholino)ethanesulfonic acid (MES) buffer (pH 6.0) was added to vial “A,” and vortexed. After approximately 3 minutes, 5 mL of MES buffer (pH 6.0) was added to vial “B” and vortexed. After approximately 2 minutes, the contents of vial “B” were added to the vial “A.” The contents of vial “A” were then stirred for approximately 5 minutes using a magnetic stir bar. The contents of tube “C” and tube “D” were then added to vial “A,” and the contents of vial “A” were stirred for approximately 90 minutes at room temperature. After the approximately 90 minutes of stirring, 320 mg of EDC was added to vial “A,” and the contents of vial “A” were stirred for an additional 90 minutes at room temperature.

After stirring, the contents of vial “A” were divided between two 50 ml conical tubes. 35 ml of ethanol was added to each conical tube, and the tubes were vortexed for approximately 1 minute, then centrifuged at approximately 2500 g for 2 minutes. The ethanol was decanted, leaving a pelletized crude product of cross-linked dextran nanoparticles in the 50-mL conical-tubes. Each pellet was dissolved with approximately 2 ml of H₂O (MilliQ), and passed through 0.22 μm centrifuge filters (Spin-X filters). The filtrate was combined into a single volume.

The crude product was visually inspected for color, transparency, and the presence of solids. The crude product was also analyzed using size-exclusion chromatography, and the size of its particles was measured using dynamic light scattering (DLS) analysis.
Particle Size Refinement

Experiments were performed to refine the size of the nanoparticles. Two approaches were used. The particle size of the crude product was refined by size-exclusion chromatography.

Alternatively, the crude product was refined by transverse flow filtration using the system shown in FIG. 2. The flow filtration was performed based on the procedure described below.

The crude product was added to a 1 L screw bottle (bottle B1), and diluted to a volume of 1 L using H₂O (MilliQ H₂O filtered through 0.22 μm filter). The bottle B1 was connected to the system, and the peristaltic pump P1 was switched on (approximately 400 RPM), forcing the crude product through a 70-kDA TFF filter. The back-pressure valve V1 was adjusted such that a pressure of approximately 23 psig was observed at the pressure gage G1. The pressure was monitored such that it did not exceed 25 psig.

Once the volume of bottle B2 reached the bottom of a feed tubing for the 10-kDA TFF filter, the second pump P2 was turned on (approximately 400 RPM), forcing the filtrate through the 10-kDA TFF filter. The back-pressure valve V2 was adjusted such that a pressure of approximately 23 psig was observed at the pressure gauge G2. The pressure was monitored such that it did not exceed 25 psig.

When the volume remaining in the bottle B1 was approximately 50 ml, the pump P1 was turned off. The collection bottle B3 was removed, and replaced with another bottle B3. The crude product in bottle B1 was diluted to a volume of 100 ml using H₂O (MilliQ H₂O filtered through 0.22 μm filter), and the pumps P1 and P2 were turned on. The pressure at gauges G1 and G2 was monitored such that it did not exceed 6.5 psig.

The size-refinement steps were repeated two times. The contents of the collection bottles were passed through 10-kDa MWCO 50 ml centrifuge filters and concentrated by centrifugation at approximately 2500 g.
DNP Labeling

Experiments were performed to label DNP with VT680-NHS (DNP-VT680) or ¹⁸F (¹⁸F-DNP). The experiments were performed based on the procedure described below.

A 1.5 ml centrifuge tube was charged with DNP-amine (8.0 mg, 100 μl), diluted with 2-(N-morpholino)ethanesulfonic acid (MES) buffer (200 μl, pH 6) and then treated with triethylamine (Et₃N, 1.5 μl) and VT680-NHS (18.5 μl, 46.4 nmol, 2.5 mM in dimethylformamide (DMF)). This was shaken at 900 rpm for 18 h at room temperature. The reaction mixture was loaded onto a PD-10 cartridge and eluted with MilliQ water (2×1000 followed by 8×500 μl fractions). Fractions 4-7 were combined and concentrated using 10-kDa MWCO filters resulting in 150 μl of concentrate.

To incorporate azides for ¹⁸F labeling via rapid click chemistry, this solution was diluted with MES buffer (200 μl) and treated with Et₃N (1.5 μl) and azidoacetic acid NHS ester (12 μl, 100 mM in DMSO) and then shaken at 900 rpm for 18 h at room temperature. This reaction mixture was loaded onto a PD-10 cartridge and eluted as described above. To end-cap remaining amines, the solution was diluted with MES buffer (200 μl) and treated with Et3N (1.5 μl) and succinic anhydride (100 μl, 750 mM in DMSO).

¹⁸F labeling of DNP was achieved by copper catalyzed azide/alkyne click chemistry (FIG. 3). First, an ¹⁸F-prosthetic group 3-(2-(2-(2-[¹⁸F]-fluoroethoxy)ethoxy)ethoxy)-prop-1-yne (¹⁸F-P3C#C) was synthesized. This was combined with the azido-DNP in the presence of copper catalyst and heated to 60° C. for 5 min. After heating, the mixture was subjected to SEC for purification. Analysis by radio thin-layer chromatography (TLC), and analytical radio-SEC were used for quality control. Success metrics include TLC analysis confirming radiochemical purity (>95% pure), confirmation of DNP identity (SEC retention time (t_{R}, ±5% of average t_{R}), and a measured specific activity equal to or greater than 10 mCi/mg DNP.
Surface Modification

Experiments were performed to cap all amines with succinic anhydride. The purpose is to lower surface change, and a lower surface charge will aid in renal clearance. In a 1.5-mL centrifuge tube, amino-DNP (DNP-NH₂, 165 μL=13.35 mg total, 3.0 μmol amines) was diluted with MES buffer (200 μL), Et₃N (2 μL) and succinic anhydride (200 μL, 750 mM in DMSO). This was shaken at 900 rpm for 18 hour at room temperature. This reaction was loaded onto a PD-10 cartridge (GE Healthcare) and eluted with MilliQ water (2×1000 μL followed by 8×500 μL fractions). Fractions 3-7 (spotted positive with 5% H₂SO₄ in EtOH on silica TLC plates) were combined and concentrated using 10-kDa molecular-weight cut-off (MWCO) filters. The contents of the filters were washed with MilliQ water (3×400 μL) resulting in 115 μL, of concentrate.

Experiments were performed to oxidize some of the dextran to form aldehydes with sodium periodate (NaIO₄). Formation of aldehydes in/on the particle allows conjugation of drugs, fluorophores for release through a pH dependent hydrolysable condensation reaction. Escalated amounts of oxidation were used to show that different amounts of drug can be conjugated to Dextran. A 50 mM solution NaIO₄ in 50 mM borate buffer was prepared. In two 1.5-mL centrifuge tubes, DNP (64 μL, 2.5 mg dextran, 6.25 mg of DNP) was transferred and labeled A and B. To A was added 11 μL of the 50 mM solution NaIO₄ and 11 μL of borate buffer. To B was added 22 μL of the 50 mM solution NaIO₄. Each tube was shaken at room temperature for 1 hour then loaded on to PD-10 columns (preconditioned with MilliQ water) and eluted with MilliQ water (2×1000 μL followed by 8×500 μL fractions). Fractions 4-9 from each reaction were combined and concentrated by 10-kDa MWCO filters each resulting in 260 μL of concentrate for Reaction A and Reaction B. This material was used for conjugation without any further purification.
Characterization

Experiments were preformed to characterize the nanoparticles. The experiments were performed based on the procedure described below.

The particle was characterized by dynamic light scattering (DLS) and zetasizer to determine size and surface charge. DLS (2.5 μl into 300 μl filtered through 0.22 um filters) and size-exclusion chromatography (SEC) serve as quality control and ensure nanoparticle integrity. The material was also analyzed by Nanodrop (2 μl into 18 μl) to quantify total moles of VT680 conjugated to the nanoparticle.

For mass quantification, a hole was punched in the top of five 1.5 ml centrifuge tubes, and the tubes were each tared. A 50 μl nanoparticle sample was added to each of the tubes, and the tubes capped. The samples were frozen in dry ice, then lyophilized. The weight of each lyophilized sample was then measured.
Content Quantification

Experiments were performed to determine the weight contribution of carboxymethyl dextran to the nanoparticles. The experiments were performed based on the procedure described below.

A stock solution of carboxymethyl dextran 4-kDa (20 mg/mL) in MilliQ water was prepared. From the stock solution, a dilution series was prepared as follows: 5, 2.5, 1.25, 0.63, 0.31, 0.16, 0.08, 0.04, and 0.02 mg/ml.

An 80% (w/w) phenol in water solution was prepared from 8 g phenol and 2 g MilliQ water.

For each concentration in the dilution series, 2.5 μl of the 80% phenol solution, 100 ul of the diluted carboxymethyl dextran, and 250 μl of concentrated H₂SO₄ were added to a respective centrifuge tube and vortexed. Each mixture was shaken at 900 RPM for 30 minutes at room temperature.

The absorbance at 490 nm of each mixture was determined using Nanodrop (2 μl/measurement). Linear regression of dilutions series data was performed, and the results were used as a standard to estimate the carboxymethyl dextran content of the nanoparticle samples.
Amine Quantification

Experiments were performed for quantifying the amine content of the nanoparticles. The experiments were performed based on the procedure described below.

50 mM glycine stock solution was prepared from 10.2 mg (0.136 mmol) of glycine dissolved in 2.72 mL of Borate buffer (50 mM). From the stock solution, a dilution series was prepared as follows: 25, 10, 5, 1, 0.1, 0.01, 0.001 and 0.0001 mM using 50 mM borate buffer.

A 20 mg/mL aminodextran (40 kDa) stock solution was prepared from 19.9 mg of aminodextran dissolved in 0.995 mL borate buffer (50 mM). From the stock solution, a dilution series was prepared as follows: 10, 5, 2.5, 1.25, 0.63, 0.31 and 0.16 mg/ml (approximately corresponding to the molarity of amines).

To separate 0.6 ml centrifuge tubes, 5 μl of each sample in the dilution series was added with SEC fraction solutions to be analyzed. 1 μl of 7.5% sodium bicarbonate, 10 μl H₂O, and 15 μl of a 30 mM TNBS solution was added to each 0.6-mL centrifuge tube. Each tube was vortexed and allowed to stand at room temperature for approximately 30 minutes.

The absorbance at 420 nm of each mixture was determined using Nanodrop (2 μl/measurement). Linear regression of dilutions series data was performed, and the results were used as a standard to estimate the amine content of the nanoparticle samples.
Azide Quantification

Experiments were performed for quantifying the azide content of the nanoparticles. The experiments were performed based on the procedure described below.

An aliquot of a nanoparticle sample was added to a centrifuge tube. 20 μl of a 25 mM FAM-5C#C solution in DMF (500 nmol), 20 μl of 80 mM BPDS solution in 1×PBS, and 20 μl of 80 mM Cu+1 solution in MeCN were added to the sample. The mixture was flushed with argon for 30 seconds and the tube was capped. The sample was microwave irradiated (60° C., 30 W for 5 min). The reaction mixture was loaded onto a PD-10 column (preconditioned with 20 mL 1×PBS), and eluted with 1×PBS. Fractions were collected (2×1000 μL, then 8×500 μL). The fractions that were yellow in color were combined, concentrated using 10-kDa MWCO filters. The material collected from the 10-kDa filters was recovered and the final volume recorded. The absorbance of FAM-5C#C conjugated to the DNP at 485 nm was measured using the Nanodrop. The concentration of FAM-5C#C from the absorbance was calculated using the equation C (aliquot)=A/eb (where e=extinction coefficient, in this case 80000).
Results

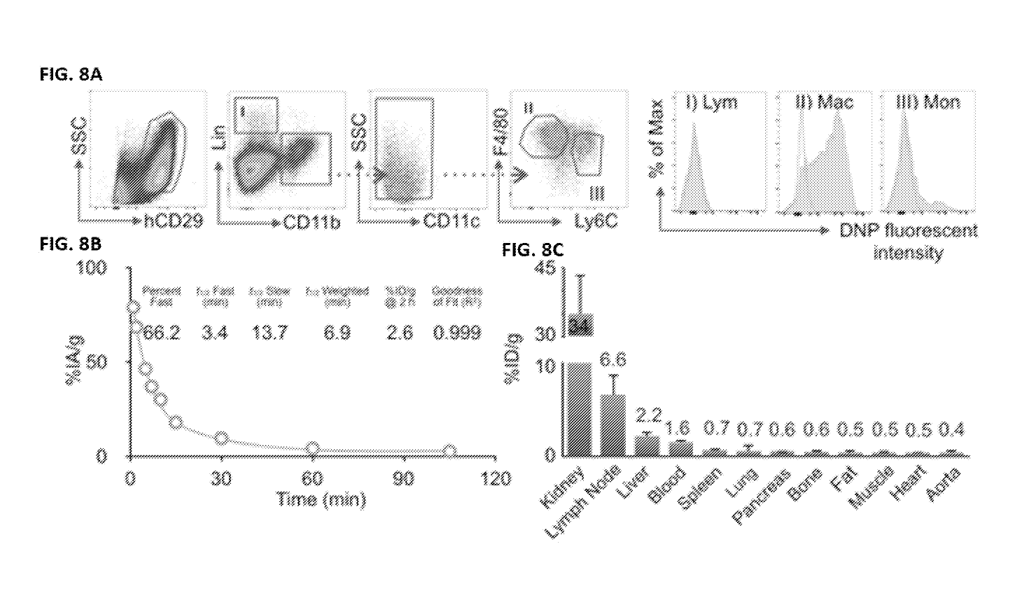

The chemical and physical properties of the carboxymethyl dextran nanoparticles in the final product were characterized based on the method described above. They are shown in Table 1 below, and in FIGS. 7A and 7B.

TABLE 1
Chemical & physical properties of carboxymethyl dextran nanoparticles

| Parameter | Value | Error (SD) |
|---|---|---|
| Diameter (DLS, nm) | | |
| Z-Average | 4.4 | 0.2 |
| Intensity | 6.4 | 0.3 |
| Volume | 4.9 | 0.4 |
| Number | 4.0 | 0.4 |
| Pdl | 0.313 | 0.054 |
| MW (kDa) | 30-50 | |
| % Dextran | 40.7 | 7.8 |
| Amine | 1.4 | 0.1 |
| (μmol/mg Dext) | | |
| Azide | 0.20 | 0.05 |
| (μmol/mg Dext) | | |
| Zeta-potential (mV) | −11.1 | 1.0 |
| [post-succinylation] | [−19.8] | [1.0] |

In addition, by increasing the amount of azides in the synthesis reaction, the amount of reactive azides in the particle was increased without affecting the key characteristics of the particle. Through these modifications, radiolabeling reactions were tested with smaller amounts of DNP. Table 2 below shows that by increasing the azide level it is possible to load a much higher quantity of ¹⁸F per milligram of nanoparticle.

### Example 2

### Blood Half-Life, Renal Clearance, and Biodistribution of DNP

Experiments were performed to determine the blood half-life, renal clearance, and biodistribution of DNP in mice and primates (Papio anubis).

### Method

Flow Cytometry

Experiments were performed to determine the distribution of DNP-VT680 in leukocytes. Mice were injected with DNP-VT680. Hearts and other organs were excised using a surgical microscope. Tissue were then minced in a mixture containing 450 U/ml collagenase I, 125 U/ml collagenase XI, 60 U/ml DNase I, and 60 U/ml hyaluronidase (Sigma) and incubated at 37° C. at 750 rpm for 1 hour. The single cell suspensions were stained with fluorochrome-labelled antibodies against mouse leukocyte lineage markers. For monocyte staining, a PE anti-mouse lineage antibody cocktail containing antibodies against CD90 (clone 53-2.1), B220 (clone RA3-6B2), CD49b (clone DX5), NK1.1 (clone PK136), Ly-6G (clone 1A8) and Ter-119 (clone TER-119) were used. Monocytes were stained with anti-mouse CD11b (clone M1/70), CD11c (clone HL3), F4/80 (clone BM8) and Ly6C (clone AL-21). These cells were then further analyzed by flow cytometry.
Biodistribution

Experiments were performed to determine the distribution of DNP in different organs. Mice were injected with ¹⁸F-DNP. Organs were harvested using a dissecting microscope and micro-dissection tools. Scintillation counting for calculating % IDGT will be recorded with a gamma counter (1480 Wizard 3″, PerkinElmer, Waltham, Mass.). Immediately after injection and again before sacrifice, all mice will be placed in a well counter (CRC-127R, Capintec, Florham Park, N.J.) to measure total corporeal radioactivity, followed by full biodistribution studies. The procedures for biodistribution studies are described in detail in Lee et al., 2012, J Am Coll Cardiol, 59, 153-63; Majmudar et al., 2013, Circulation, 127, 2038-46; Majmudar et al., 2013, Circ Res, 112, 755-61. They are herein incorporated by reference.
Blood Clearance

Experiments were performed to determine the clearance rate of DNP in the blood. A subject was injected with DNP. The amount of DNP in the blood at different time points was measured and determined. C57BL6 mice were used for blood half-life determinations. Mice were administered 50±5 μCi of ¹⁸F-DNP by intravenous tail vein injection. Blood sampling was performed by retro-orbital puncture using tared, heparinized capillary tubes. Samples were subsequently weighed, and activity was measured using an automatic gamma counter (Wallac Wizard 3″ 1480 Automatic Gamma Counter; PerkinElmer, Waltham, Mass.). Blood half-life data were fitted to a biexponential model (Graph-Pad Prism 4.0c; GraphPad Software, Inc, San Diego, Calif.), and results were reported as the weighted average of the distribution and clearance phases.

### Results

Blood Half-Life and biodistribution in mice

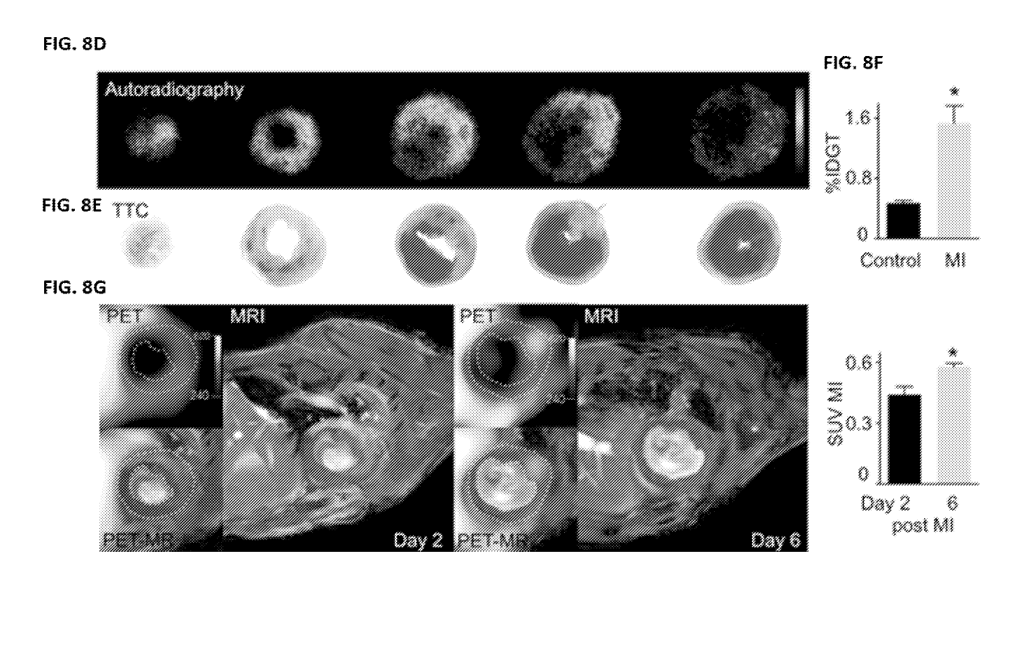

Mice were injected with DNP-VT680. The hearts and other organs were excised and used for flow cytometry analysis and biodistribution analysis. The results showed the DNP particles were mainly phagocytized by macrophages (FIG. 8A).

The amount of the DNP in blood was also measured. The blood counts were further fit to a two-phase exponential decay with a 3.4 min half-life for the initial distribution phase and 13.7 min for the elimination phase (FIG. 8B).

Mice were injected with ¹⁸F-DNP. Organs were harvested using a dissecting microscope and micro-dissection tools. Experiments were performed to determine the distribution of DNP in different organs. The result is shown in FIG. 8C. Excised hearts were sectioned and treated with 1% (w/w) triphenyltetrazolium (TTC) for 15 min to visualized infarcted tissue (FIG. 8E, white colored tissue). The sections were mounted and exposed on an autoradiography plate overnight. The of the scanned autoradiography plate are shown in FIG. 8D (color scale: red indicates high uptake of 18F-DNP, black indicates no uptake). Comparison of TTC staining and autoradiography demonstrates good uptake of 18F-DNP in infarcted heart tissues. Ex vivo counting of infarcted and healthy hearts from mice systemically injected with equal doses of ¹⁸F-DNP shows a greater than 3-fold higher uptake of ¹⁸F-DNP in infarcted tissue (FIG. 8F).

### Renal Clearance in Primates (Papio anubis)

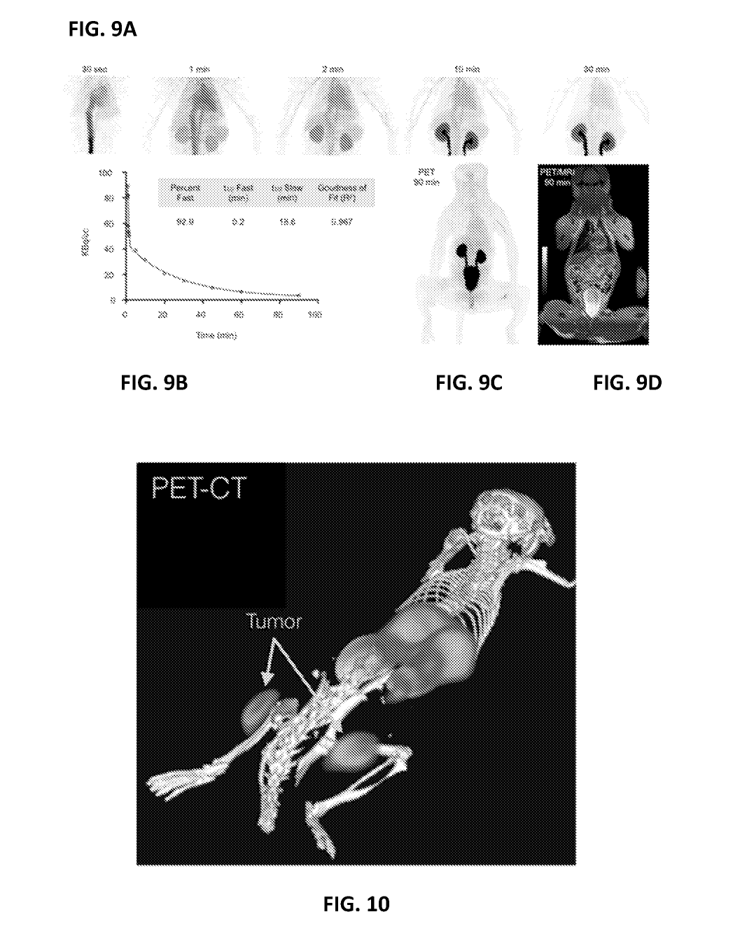

¹⁸F-DNP was injected to Papio Anubis. The result for the PET/MRI in a baboon (Papio anubis) showed a rapid renal clearance of ¹⁸F-DNP from the blood pool (FIGS. 9A, 9C, 9D). Blood counts were further fit to a two-phase exponential decay with a 0.2 min half-life for the initial distribution phase and 18.6 min for the elimination phase (FIG. 9B). The data indicated that ¹⁸F-DNP has rapid renal clearance in primates.

### Example 3

### Imaging and Quantifying Macrophages In Vivo

Experiments were performed to image and quantify macrophages in vivo.

### Methods

Animal Models

Mouse MI model: B6.129P2-Apoe^{tm1Unc}/J (ApoE^{-/-}) mice were purchased from Jackson Laboratory. ApoE^{-/-}mice were treated with a high-cholesterol diet (Harlan Teklad, 0.2% total cholesterol) for 10 weeks before the experiments began. ApoE^{-/-} mice were used for infarct studies because they have pre-existing atherosclerosis and therefore better resemble the clinical scenario of acute MI. Mice were then injected with buprenorphine (0.1 mg/kg i.p.), then anesthetized with isoflurane and ventilated with 2% isoflurane supplemented with 02. Thoracotomy were performed in the fourth left intercostal space. The left coronary artery was permanently ligated with a nylon 8-0 suture. Mice were then treated with buprenorphine for 3 days (twice daily 0.1 mg/kg i.p.). Analgesia and anesthesia were used (buprenorphine 0.1 mg/kg i.p. and ventilation with 2% isoflurane/O₂).

Mouse tumor model: HT1080 (fibrosarcoma, human) cells are injected into Nu/Nu mice. Other cancer models used were: Panc02 (pancreatic adenocarcinoma, mouse) cells were injected into Nu/Nu mice, and B16 (melanoma, mouse) cells were injected in to C57BL6 mice. The mice received subcutaneous injections, into their flanks (2.5×10⁶ cells in 100 μl of 70:30 PBS/BD Matrigel [BD Biosciences, Bedford, Mass.] per injection). Tumors were then allowed to grow for 2 weeks before imaging. For dose-response experiments, nu/nu mice each received two subcutaneous injections containing A2780 cells into the flanks (2.5×10⁶ cells in 100 μl of 70:30 PBS/BD Matrigel [BD Biosciences] per injection). Tumors were then allowed to grow for 10 to 15 days before the start of imaging experiments.
PET/MRI and PET/CT

Dynamic mouse PET were performed on a Siemens Inveon PET-CT system. Mice were anesthetized by isoflurane prior to imaging, and anesthesia was maintained via a nosecone. CT acquisition preceded PET and lasted approximately 4 minutes, acquiring 360 cone beam projections with a source power and current of 80 keV and 500 μA. respectively. Projections were reconstructed into three-dimensional volumes containing 512×512×768 voxels with the dimensions 0.11×0.11×0.11 mm. The imaging bed then moved into the PET gantry, The radioactive agent was injected at the beginning of PET acquisition via tail vein catheter, which was set up prior to imaging. A high-resolution Fourier re-binning algorithm was used to re-bin sinouarns, followed by a filtered back-projection algorithm for reconstruction. Image voxel size is 0.80×0.86×0.86 mm. The reconstructed PET image, through dynamic framing of the sinograms was composed of a series of 1, 3 and 5 minute frames. PET and CT reconstructed images were then fused using Inveon Research Workplace (IRW) software (Siemens). Regions of interest were drawn in IRW to calculate data as mean standardized uptake values (SUV) or view kinetic analysis of dynamic PET data.

Mice were imaged using 2 separate systems, the Inveon and a 7 Tesla Bruker. PET/MRI registration and fusion are facilitated by a custom-made mouse bed and PET-CT gantry adapter as described in Lee et al., 2012, J Am Coll Cardiol, 59, 153-63. We implemented a fusion approach using external fiducial landmarks, provided by a “vest” optimized for cardiac imaging. The vest surrounds the mouse's chest to create a frame that follows minor movements due to transfer between scanners or light anesthesia. The tubes are filled with 15% iodine in water, rendering them visible in both CT and MRI. Mouse motion was minimized with an imaging bed that can be used in both imaging systems. Diastolic PET data were pre-fused to CT as part of a standard workflow. This protocol was validated using the cross correlation function on phantom images and had been used by us for vascular and myocardial PET/MRI. The details were described in Lee et al., 2012, J Am Coll Cardiol, 59, 153-63; Majmudar et al., 2013, Circulation, 127, 2038-46; Majmudar et al., 2013, Circ Res, 112, 755-61. They are herein incorporated by reference.

### Results

The PET/MRI results for the mice MI model were analyzed. The results show that ¹⁸F-DNP uptake in the infarct, reflecting increasing macrophage numbers in this area (FIG. 8G).

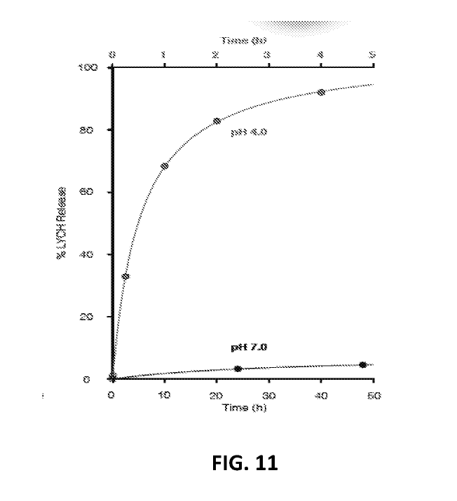

Experiments were also performed to determine whether ¹⁸F-DNP can target macrophages around tumor cells. The PET/CT results for the mice tumor model showed that the ¹⁸F-DNP allowed for macrophage-specific PET-CT imaging of bilateral flank tumors (FIG. 10).

### Example 4

### DNP Conjugates

Experiments were performed to determine whether the DNP particle can deliver drug to a target site. For testing, we used a fluorescent dye, lucifer yellow carbohydrazine and the chemotherapeutic doxorubicin.

Experiments were performed to synthesize CMDex-LY (FIG. 5). CM-Dextran was first treated with NaIO₄ (in 0.9 μmol, 2.2 μmol, and 4.4 μmol) and then 250 nmol Lucifer Yellow (LYCH). Three test reactions under different conditions were performed. The result and the conditions were shown in Table 3 below.

TABLE 3
Results and Reaction Conditions for Synthesizing CMDex-LY

| Expt | | 1 | 2 | 3 |
|---|---|---|---|---|
| NalO₄ | CMDex (mg) | 10 | 10 | 10 |
| Oxidation | NalO₄ | 0.9 | 2.2 | 4.4 |
| | (μmol) | | | |
| LYCH | CMDex (mg) | 1.0 | 1.0 | 1.0 |
| Conjugation | LYCH (nmol) | 250 | 250 | 250 |
| | LY (nmol) | 91.5 | 154 | 204 |
| | Conjugated | | | |
| | LY/CMDex | 91.5 | 154.0 | 204.0 |
| | (nmol/mg) | | | |

Experiments were performed to synthesize DNP-LY (FIG. 5). Two test reactions under different conditions were performed. In a 1.5-mL centrifuge tube, DNP-CHO #1A (104 μL, 1 mg dextran, 2.5 mg DNP) was diluted with an equal volume of 50 mM sodium acetate buffer (pH 7.5) and 50 mM Lucifer Yellow CH solution (10 μL). In a separate 1.5-mL centrifuge tube, DNP-CHO #1B (104 μL, 1 mg dextran, 2.5 mg DNP) was diluted with an equal volume of 50 mM sodium acetate buffer (pH 7.5) and 50 mM Lucifer Yellow CH solution (10 μL). These two tubes were placed on an orbital shaker. After 24 h, the reaction mixtures were transferred to PD-10 columns (preconditioned with MilliQ water) and eluted with MilliQ water (2×1000 μL followed by 8×500 μL fractions). Separately, fractions 3-8 from each reaction were combined and concentrated by 10-kDa MWCO filters resulting in 270 and 310 μL for Reaction A and B, respectively. An aliquot from each reaction was removed and diluted by a factor of 2 with 50 mM sodium acetate buffer (pH 7.5) and analyzed by Nanodrop (430 nm) to determine concentration. Reaction A was shown to have 29.3 nmol LY/mg DNP and Reaction B was shown to have 42.7 nmol LY/mg DNP. The result and the conditions were shown in Table 4 below.

TABLE 4
Results and reaction conditions for synthesizing DNP-LY

| Expt | | 1 | 2 |
|---|---|---|---|
| NalO₄ | mg DNP | 6.27 | 6.27 |
| Oxidation | mg Dextran | 2.50 | 2.50 |
| | NalO₄ | 0.5 | 1.1 |
| | (μmol) | | |
| LYCH | DNP (mg) | 2.51 | 2.51 |
| Conjugation | Dextran | 1.0 | 1.0 |
| | (mg) | | |
| | LYCH | 500 | 500 |
| | (nmol) | | |
| | LY (nmol) | 73.5 | 107.3 |
| | Conjugated | | |
| | LY/DNP | 29.3 | 42.7 |
| | (nmol/mg) | | |

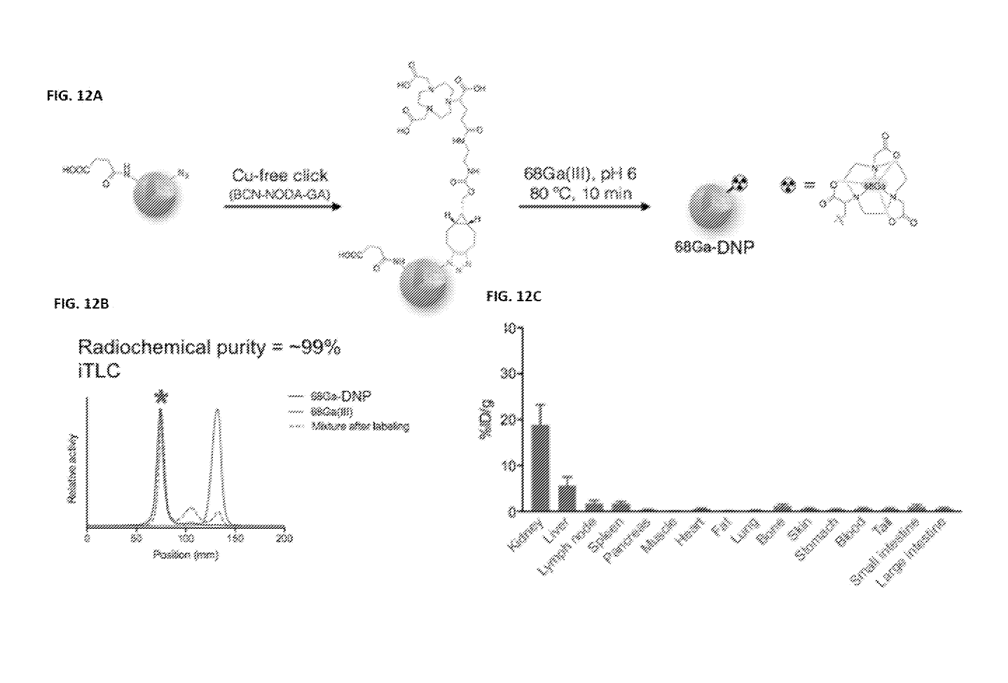

Experiments were performed to determine whether the DNP-LY can release LYCH when pH is low. The result showed more than 90% of LYCH was released in 4 hours when pH was 4.0. In contrast, less than 10% of LYCH was released in 2 days (FIG. 11).

Experiments were performed to determine whether the DNP can deliver a drug that is conjugated to DNP nanoparticle by hydrazine to macrophages in vivo. An azide reactive (SPAAC) VT680XL reagent was prepared so that the fluorescent DNP and lucifer yellow (LYCH) can be tracked in vivo. Thus, the DNP-LY-680 nanoparticle was labeled with both LYCH and VT680XL. A single normal mouse was injected with DNP-LY-680 as well as fluorescent F4/80 antibody. One hour later the mouse was euthanized and heart was excised and imaged. The result showed that colocalization of fluorescent DNP and Lucifer yellow, demonstrating delivery of DNP payload to macrophages.

Experiments were performed to conjugate DNP particles with doxorubicin as shown in FIG. 6. Test reactions under different conditions were performed. The result and the conditions were shown in Table 5 below.

TABLE 5
Results and Reaction Conditions for Synthesizing DNP-Doxorubicin

| Expt | | 1 | 2 |
|---|---|---|---|
| Boc | mg DNP | 4.38 | 4.38 |
| Deprotection | HCl (M) | 0.75 | 1.5 |
| | Time (h) | 0.5 | 2.0 |
| Dox | mg DNP | 4.18 | 4.18 |
| Conjugation | Dox (nmol) | 250 | 250 |
| | nmol Dox | 37 | 118 |
| | Conjugated | | |
| | Dox/mg DNP | 8.9 | 28.2 |

### Example 5

### Quantifying Macrophages in Large Mammals

Experiments are performed to determine whether ¹⁸F-DNP PET imaging is suitable for quantifying macrophages in large mammals (and by extension humans) with ischemic heart disease.

### Method

Swine Model of MI

Left anterior descending coronary artery (LAD) balloon occlusion is used to induce MI. Pigs are allowed to acclimate for a week prior to MI, which are induced under general anesthesia following sedation with 4.4 mg/kg telazol and 2.2 mg/kg xylazine intramuscularly. Once sedated, a 22 or 20 g intravenous catheter (IVC) is placed in an ear vein using aseptic technique. Isoflurane (1-3%) is used for inhalation anesthesia. Prior to MI induction, animals receive buprenorphine analgesia (0.1 mg/kg s.c.) to alleviate pain from ischemia. The same analgesia is given twice daily for 48 hrs after surgery. Pigs are intubated and ventilated at a rate of 12 per min. 20,000 IU of heparin is injected i.v. to avoid thrombotic complications. Local anesthesia (Lidocaine 0.5% s.c.) precedes placing a sheath in the right carotid artery. A 7-F guiding catheter is advanced through the introducer sheath using the Seldinger technique. A guide wire is placed into the LAD using X-ray guidance. An angioplasty balloon is advanced to a position distal to the first diagonal artery using X-ray fluoroscopy guidance and inflated for 60 minutes to induce large infarcts with the potential for adverse left ventricular remodeling.

Angiography confirms complete occlusion of the vessel. If ventricular fibrillation occurs, pigs will be defibrillated. Pigs are checked twice daily for failure to feed, heart murmurs, and loss of body weight.
PET/MRI for Large Animal

Large animal PET/MRI is conducted. ¹⁸F-DNP are injected while the pig is positioned in the scanner's bore. Pigs are then anesthetized, and a veterinarian supervises anesthesia during imaging. Continuous PET imaging is performed for 120 min over a 25 cm field of view that includes the heart. Regions of interest are drawn in the myocardium identified by MRI as described below. Tracer accumulation is modeled using a 2-compartment model (pMOD Cardiac, Zurich). The arterial input function is sampled to allow full pharmacokinetic tracer modeling in the infarct, border zone and remote zone. MRI data is acquired using a two-point Dixon approach to perform attenuation correction. 0.2 mmol/kg of Gd-DTPA (Magnevist, Schering) is injected and we perform delayed-gadolinium-enhancement imaging 10-15 min later with a 2D inversion recovery gradient echo approach. The inversion time will be optimized to null the myocardium. During the washout of Gd-DTPA, LV function will be imaged using a bSSFP cine sequence with rate 2 acceleration. The necessary procedures for PET/MRI is described in Ye et al., 2015, Circ Res, 117, 835-45. It is herein incorporated by reference.

DNP should behave in the swine model in fundamentally the same manner as they do in similar experiments in mice and primates and thus the swine tests should confirm that ¹⁸F-DNP can quantify inflammatory macrophages in cardiovascular tissues in large-animal models of ischemic heart disease. This experiment is an important step for translating macrophage-specific PET imaging into the clinic.

### Example 6

### Imaging Mice using ⁶⁸Ga Labeled DNP

Experiments were performed to image mice using ⁶⁸Ga labeled DNP.

The method of labeling of DNP with the PET radioisotope 68-gallium (⁶⁸Ga) is shown in FIG. 12A. DNP (100 μg, ˜240 nmol of azide/mg) was dissolved with 200 μL of deionized water. NODA-GA was linked to DNP-azides via Cu-free strain-promoted alkyne-azide cyclization (SPAAC). BCN-NODA-GA (CheMatech, Dijon France, Cat. # C131, bicyclononyne-1,4,7-triazacyclononane,1-glutaric acid-4,7-acetic acid) (3 mg, 5.1 μmol) was added to the DNP aqueous solution followed by agitation at room temperature overnight. Unreacted BCN-NODA-GA was removed by PD-10 column purification, and water was used as an eluent. Dextran-positive fractions were combined and centrifuged (10 kDa Molecular weight cut-off (MWCO)) to give a final volume of ˜500 μL. Light yellow solution can be lyophilized, resulting in beige solid, and stored for future use.

The NODA-DNP was labeled with 1-2 mCi (˜60 MBq) ⁶⁸Ga (with the concentration around 500 pM) under the condition pH=6, 80° C. for about 10 minutes. FIG. 12B shows the radiochemical purity of ⁶⁸Ga labeled DNP as determined by instant thin-layer chromatography (iTLC).

⁶⁸Ga labeled DNP were administered to wild type mice and ApoE^{-/-} mice with atherosclerosis. The PET imaging was performed on these mice. Data obtained from wild type mice indicated that ⁶⁸Ga-DNP is excreted through kidney (FIG. 12C). Data obtained from ApoE^{-/-} mice with atherosclerosis demonstrated that ⁶⁸Ga-DNP enriches in atherosclerotic plaques, which are known to be full of macrophages. FIG. 12D shows autoradiography of aorta harvested from ApoE^{-/-} mice with atherosclerosis. The area with strong signal co-localized with plaque stained red by Oil Red O. FIG. 12E shows high signal in kidneys. The result is consistent with renal excretion of ⁶⁸Ga-DNP. FIG. 12F shows increased PET signal in the aortic root of an ApoE^{-/-} mice with atherosclerosis. The results demonstrate that ⁶⁸Ga labeled DNP can be used as a PET agent for in vivo imaging.

### OTHER EMBODIMENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

1. A nanometer-sized dextran nanoparticle comprising a plurality of carboxymethyl dextran polymer chains and lysine, wherein the carboxymethyl dextran polymer chains are cross-linked by lysine.

2. The dextran nanoparticle of claim 1, wherein the size of the dextran nanoparticle is between about 3 nm and about 15 nm.

3. The dextran nanoparticle of claim 1, wherein the size of the dextran nanoparticle is greater than 3 nm and less than 10 nm.

4. The dextran nanoparticle of claim 1, wherein the dextran nanoparticle further comprises a functional group that optionally links the nanoparticle to an active agent.

5. The dextran nanoparticle of claim 4, wherein the functional group is an azide functional group.

6. The dextran nanoparticle of claim 4, wherein the functional group is a sulfonate functional group.

7. The dextran nanoparticle of claim 4, wherein the functional groups are selected from amino, —NHC(O)(CH₂)ₙC(O)—, carboxy, and sulfhydryl groups.

8. The dextran nanoparticle of claim 4, wherein the active agent is —(CO)ₙ-¹⁸F, wherein n is from about 0 to about 5.

9. The dextran nanoparticle of claim 4, wherein the active agent is ¹⁸F or ⁶⁸Ga.

10. The dextran nanoparticle of claim 4, wherein the active agent is a fluorophore.

11. The dextran nanoparticle of claim 10, wherein the fluorophore is VT680 or VT750.

12. The dextran nanoparticle of claim 10, wherein the fluorophore is BodipyFL or Bodipy630.

13. The dextran nanoparticle of claim 4, wherein the active agent is a drug.

14. A dextran nanoparticle composition comprising a plurality of dextran nanoparticles according to claim 1, wherein the average largest diameter of the plurality of the nanoparticles is between about 3 nm and about 15 nm.

15. The composition of claim 14, wherein more than 95% of the nanoparticles in the composition have a diameter between 3 nm and 15 nm.

16. The composition of claim 14, wherein more than 95% of the nanoparticles in the composition have a diameter between 4 nm and 7 nm.

17. An in vivo method of imaging macrophages in a subject, the method comprising:
1) administering to the subject an effective amount of dextran nanoparticles according to claim 1, wherein the dextran nanoparticles comprise an imaging agent linked to the nanoparticles; and
2) after a suitable waiting period, imaging the imaging agent in a region of the subject in which the macrophages have accumulated using an imaging technique.

18. The method of claim 17, wherein the imaging technique is PET.

19. The method of claim 17, wherein the imaging technique is PET/CT.

20. The method of claim 17, wherein the imaging technique is PET/MRI.

21. A method of delivering a therapeutic agent to a target site in a subject, the method comprising administering to the subject an effective amount of dextran nanoparticles according to claim 1, wherein the dextran nanoparticles further comprise a therapeutic agent linked to the dextran nanoparticle.

22. The method of claim 21, wherein the therapeutic agent is doxorubicin.
